(19) United States
(12) Patent Application Publication
(10) Pub. No.: US 2021/0112819 A1
(43) Pub. Date: Apr. 22, 2021

(54) MEAT INJECTION DEVICE WITH A SELF-ADJUSTING POSITION REFERENCE AND AUTOMATIC REFILL

(71) Applicant: Hugh Hetherington, Bozeman, MT (US)

(72) Inventor: Hugh Hetherington, Bozeman, MT (US)

(21) Appl. No.: 17/137,162

(22) Filed: Dec. 29, 2020

Related U.S. Application Data
(63) Continuation-in-part of application No. 15/608,861, filed on May 30, 2017.
Application No. 15/608,861 is a continuation-in-part of application No. 14/025,933, filed on Sep. 13, 2013, now abandoned.
Application No. 14/025,933 is a continuation-in-part of application No. 13/734,974, filed on Jan. 5, 2013, now abandoned.
Application No. 13/734,974 is a continuation-in-part of application No. 12/285,203, filed on Sep. 30, 2008, now abandoned.
Application No. 12/285,203 is a continuation-in-part of application No. 12/078,603, filed on Apr. 2, 2008, now Pat. No. 8,133,208.

(51) Int. Cl. A22C 17/00; A23B 4/28

(52) U.S. Cl. CPC A22C 17/0053; A22C 9/001; A23V 2002/00; A23B 4/28

(57) ABSTRACT

An automatic injection control device injects fluid at a rate directly related to the displacement rate of the device body from a held positioning member. The device has a syringe end, a gripping end, a syringe barrel coupled to the body and a refillable reservoir and at least one directional valve at the syringe end of the body. The directional valves permitting refilling of the refillable reservoir without removing the syringe barrel. Also, a transmission system is coupled to the body, as well as the positioning member, a syringe plunging member, and a gearing mechanism linking the positioning member to the syringe plunging member. The device is designed so that when the positioning member is held in a fixed extended position and the body is pulled by the gripping end, the transmission system provides a one-way motion of the syringe plunging member to precisely inject fluid from the device.

### CROSS REFERENCE TO RELATED APPLICATIONS

This application is a Continuation-In-Part of pending U.S. patent application Ser. No. 15/608,861 filed May 30, 2017, which is a Continuation-in-Part of U.S. patent application Ser. No. 14/025,933 filed Sep. 13, 2013 (and now abandoned), which is a Continuation-In-Part of U.S. patent application Ser. No. 13/734,974 filed Jan. 5, 2013 (and now abandoned), which is a Continuation-In-Part of U.S. patent application Ser. No. 12/285,203 filed Sep. 30, 2008 (and now abandoned) which is a Continuation-In-Part of U.S. patent application Ser. No. 12/078,603, filed Apr. 2, 2008, issued as U.S. Pat. No. 8,133,208 on Mar. 13, 2012, and claims benefit to the priorities thereof. The contents being incorporated herein by reference in their entirety.

### FIELD

This disclosure relates to an injection device, referred to hereafter as the injection control device (ICD). More particularly, this disclosure relates to a design that allows the automatic injection (into subject material), a proportional volume of solution or injectate from the syringe as a function of cannula displacement, while also automatically refilling the ICD syringe from a separate injectate-filled feed vial or feed tube.

### BACKGROUND

In the meat preparation arts, as one example, there is the requirement for placement of material, for example, a brine or flavored solution into a meat in a manner that leaves a defined thickness or consistent profile of material per unit region. That is, the channel created by the cannula's insertion (extraction) is filled with the filler material. To obtain a consistent moisture within the meat and saturation of flavor within (from the injectate solution), the user must move the cannula at a precise rate (aka—a pass) while coordinating the rate of injecting the material to match the cannula's rate of movement, hopefully avoiding causing lumping (too much) or sinking (too little) in a region. Too little or too much displacement of material (or inconsistent cannula movement) causes overfilling or underfilling of the solution, which affects the end flavor of the meat.

In particular, the traditional method is to manually withdraw or inject the cannula of the syringe into the meat while “manually” pressing (with the thumb) the syringe's plunger in synchronicity. Of course, it goes without saying this approach is sensitive to the user's skill level and produces different results for different passes. Further, the effort of manual pressing for each pass can be fatiguing. Being unavoidably subject to human error, inconsistent results (e.g., lumps, voids, etc.) often occur. Accordingly, there has been a long-standing need in the food discipline to devise systems and methods for addressing the problems discussed above.

### SUMMARY

The following presents a simplified summary in order to provide a basic understanding of some aspects of the claimed subject matter. This summary is not an extensive overview and is not intended to identify key/critical elements or to delineate the scope of the claimed subject matter. Its purpose is to present some concepts in a simplified form as a prelude to the more detailed description that is presented later.

In accordance with one aspect of the present disclosure, an injection control device is provided, comprising: a body having a syringe end and a gripping end; a syringe barrel coupled to the body; a refillable reservoir and at least one directional valve, disposed at the syringe end of the body, one of the at least one directional valves permitting refilling of the refillable reservoir without removing the syringe barrel; and a transmission system coupled to the body, comprising: a positioning member coupled to the transmission system and extendable from the syringe end of the body; a syringe plunging member coupled to the transmission system; and a gearing mechanism linking the positioning member to the syringe plunging member, wherein when the positioning member is held in a fixed position and the body is pulled by the gripping end, the transmission system provides a one-way motion of the syringe plunging member to inject fluid from the device, a rate of the injected fluid being directly related to a rate of displacement of the body from the held positioning member.

In accordance with another aspect of the present disclosure, the injection control device of above is provided, wherein the refillable reservoir is automatically refilled with injectate if the one of the at least one directional valves is connected to an injectate supply as the positioning member is returned to a resting, non-extended state; and/or further comprising a cannula coupled to the syringe end; and/or wherein the gearing mechanism comprises a plurality of gears, at least one gear contacting the positioning member and at least one other gear contacting the syringe plunging member; and/or further comprising, a retraction mechanism coupled to the syringe plunging member or to a piston within the refillable reservoir, configured to provide a returning action to the positioning member upon its release from being held, wherein the returning action engages the transmission system to withdraw the syringe plunging member and automatically refill the refillable reservoir from injectate entering the one of the at least one directional valves; and/or wherein the retraction mechanism is a spring; and/or where another of the at least one directional valves is configured to prevent injectate from being drawn back into the refillable reservoir from the cannula; and/or wherein the another of the at least one directional valves only provides flow from the refillable reservoir to the cannula; and/or, wherein the one of the at least one directional valves is displaced from the refillable reservoir and connected to a refill tube; and/or further comprising a container of injectate connected to a refill tube connected to the at least one directional valve, wherein the container is either pressurized or non-pressurized; and/or further comprising a refill vial, coupled directly or indirectly to the one of the at least one directional valves; and/or wherein the at least one directional valves and switchable from bi-directional to unidirectional; and/or further comprising a movable piston within the refillable reservoir and coupled to the syringe plunging member.

In accordance with yet another aspect of the present disclosure, an injection control device is provided, comprising: a body having a syringe end and a gripping end; a syringe barrel coupled to the body; one or more injectate-refillable means disposed proximal to the syringe end of the body; a flow control means coupled to the injectate-refillable means; a transmission means coupled to the body, comprising: a positioning means coupled to the transmission means and extendable from the syringe end of the body; a syringe plunging means coupled to the transmission means; and a linking means linking the positioning means to the syringe plunging means, wherein when the positioning means is held in a fixed position and the body is pulled by the gripping end, the transmission means provides a one-way motion of the syringe plunging means to inject fluid from the device, a rate of the injected fluid being directly related to a rate of displacement of the body from the held positioning means.

In yet another aspect of the disclosure, the above device is described wherein the injectate-refillable is automatically refilled with injectate if the one of the at least flow control means is connected to an injectate supply as the positioning means is returned to a resting, non-extended state; and/or wherein the linking means contacts the positioning means and the syringe plunging means; and/or further comprising, a retraction means coupled to the syringe plunging means or to the injectate-refillable means, configured to provide a returning action to the positioning means upon its release from being held, wherein the returning action engages the transmission means to withdraw the syringe plunging means and automatically refill the injectate-refillable means from injectate entering the flow control means; and/or another flow control means disposed at the syringe end of the body to prevent injectate from being drawn back into the injectate refillable means from a cannula.

In yet another aspect of the disclosure, a method for refilling an injection control device without replacing a syringe barrel is provided, comprising: coupling a refillable reservoir and at least one directional valve, to a syringe of an injection control device body having a syringe end and a gripping end; coupling a transmission system to the body; coupling a positioning member to the transmission system, the positioning member being extendable from the syringe end of the body; coupling a syringe plunging member to the transmission system, the syringe plunging member be movable by the transmission system; linking a gearing mechanism to the positioning member and to the syringe plunging member; and refilling the refillable reservoir by retracting the positioning member, a motion of the positioning member operating on the transmission system to cause the syringe plunging member to retract from the refillable reservoir and create a suction to cause injectate to flow into the refillable reservoir from an external source coupled to the at least one directional valve.

In yet another aspect of the disclosure, the above method is provided, further comprising coupling a retraction mechanism to at least one of the syringe plunging member and refillable reservoir, to automatically cause the positioning member to retract back to a non-extended state, wherein the refillable reservoir is filled from an external pressurized or non-pressurized injectate container.

### BRIEF DESCRIPTION OF THE DRAWINGS

FIG. 1 is an illustration of an exemplary injection control device (ICD) with a syringe.

FIG. 2 is an illustration of the side view of a separated exemplary ICD of FIG. 1.

FIG. 3 is a cut-away illustration of the embodiment of FIG. 1, showing the internal components of the ICD.

FIG. 4 is a closeup reverse illustration of the interior of the exemplary ICD.

FIG. 5 is a bottom side illustration the exemplary ICD with the syringe rack removed from view.

FIG. 6 is a perspective illustration of a syringe rack arrangement of an exemplary ICD.

FIG. 7 is an illustration of an exemplary ICD with multiple gears viewable.

FIG. 8 is an illustration of a perspective bodiless view of a “rackless” exemplary ICD according to a second exemplary embodiment.

FIG. 9 is an illustration of a perspective bodiless view of another “rackless” exemplary ICD according to a third exemplary embodiment.

FIG. 10 is an illustration of a bodiless cut-away view of the exemplary ICD of FIG. 9. [

FIG. 11 is an illustration of a “bodied” cut-away view of the exemplary ICD of FIG. 9 with adjustable stops.

FIG. 12 is an illustration of a perspective view of an exemplary ICD according to a fourth exemplary embodiment.

FIG. 13 is an illustration of another exemplary injection control device according to a fifth embodiment, designed for automatic or near-automatic refilling.

FIG. 14 is a side cut-away view of the embodiment of FIG. 13, shown in an extended state (post-use) with a cannula attached to the syringe system

FIG. 15 is a blow up illustration of the mechanics of a commercially available pistoned chamber and refill canister system suitable for the exemplary ICD of FIGS. 13-14.

FIG. 16 is a blow up illustration of another commercially available pistoned chamber with a refill tube system suitable for the exemplary ICD of FIGS. 13-14.

### DETAILED DESCRIPTION OF THE DRAWINGS

The claimed subject matter is now described with reference to the drawings. Reference to the above incorporated application(s)/patent(s) provide extensive descriptions on the mechanics for achieving semi-automatic, rate-specific delivery/extraction. In particular, the incorporated embodiments are shown where, in most instances, the positioning guide is manually extended/operated to establish a reference position for when the cannula and plunger are simultaneously operated in synchronicity (i.e., coordinated via mechanical means to move in direct proportion to each other). For example, in an injection scenario, the positioning guide is manually “held” in position “against” the tissue, by the user while the injection control device (ICD) is being pulled away (toward) during operation. Further, upon completion of the “injection” the device's syringe barrel must be manually refilled, typically by removing the spent barrel and inserting a “fresh” barrel with injectate material.

In various embodiments as now described in this disclosure, the configuration of the injection control device (ICD) is such that the injectate is automatically drawn into the ICD's syringe barrel or proxy to it from a separate larger container of injectate. A flow-controlled valve controls the entry of the injectate into the syringe barrel (or proxy), thus enabling rapid refilling of the syringe barrel, without resorting to the removal and insertion of the syringe barrel from the ICD Thereby, a user can simply “inject” and re-inject in rapid succession since the syringe barrel or poxy to it is automatically refilled.

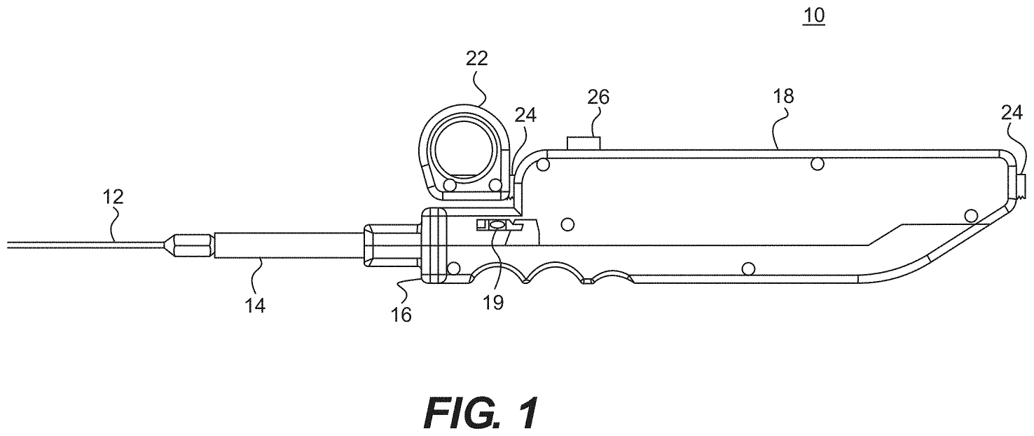

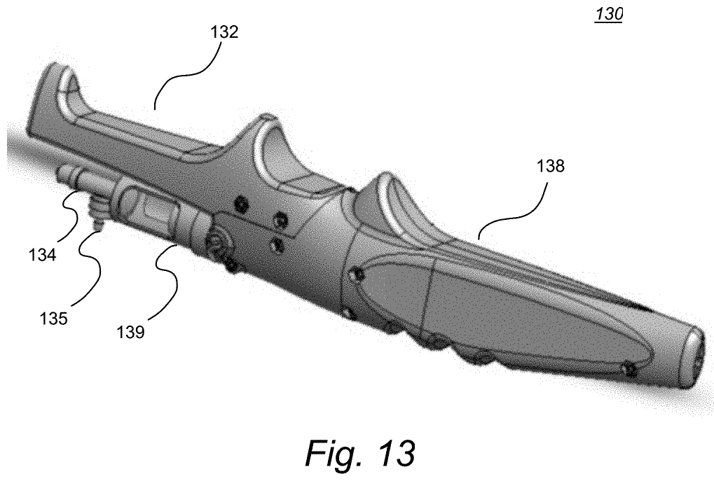

Aspects of the operation of a simple ICD's controlled injection scheme (e.g., consistent rate/volume of injectate per cannula movement rate) is described in the following FIGS. 1-12 while the operation of a stream-lined automatically refilling ICD is described in FIGS. 13-16.

FIG. 1 is an illustration of a side view 10 of an exemplary injection control device according to an embodiment of the invention. The exemplary injection control device is illustrated with a cannula or needle 12 coupled to a cannula mating section 14. It should be apparent that the cannula 12 may be removable or be of a disposable form. The cannula mating section 14 may be, in some instances, referred to as the syringe of the exemplary injection control device. The syringe 14 may be configured to be supported and/or held securely by a syringe-supporting section 16 of the body 18. The syringe 14 may also be disposable, if so desired, and may be configured in varying sizes, according to design or application preference. Accordingly, the syringe supporting section 16 may be configured to be adapted to various shapes or sizes of the syringe 14, according to design or application preference. While the cannula 12 is illustrated as having a straight shape, other curvatures or shapes may be used according to application preference.

The body 18 is illustrated as containing a latch 19 which operates to secure the upper and lower portions of the body 18, during assembly. The body 18 accommodates an exposed ring 22 which is connected to a positioning rack 24 (partially obscured) which is housed or protected by the body 18. The positioning rack 24 is shown in FIG. 1 as being situated to travel through the body 18 and is subject to engagement of the brake 26. In some embodiments, the positioning rack 24 may be placed exterior of the body 18, according to design preference, such as, for a non-limiting example, a sliding arrangement as seen in older slide rules. The brake 26 operates to prevent travel of the positioning rack 24 when engaged, or conversely, when dis-engaged, depending on design implementation.

While FIG. 1 illustrates the exposed ring 22 as being circular in shape, it should be understood that other shapes, closed or open, may be used without departing from the spirit and scope of this disclosure. In fact, in some embodiments, it may be desirable to have a “flat” surface or “plate” rather than the exposed ring 22, depending on the user's preference or application.

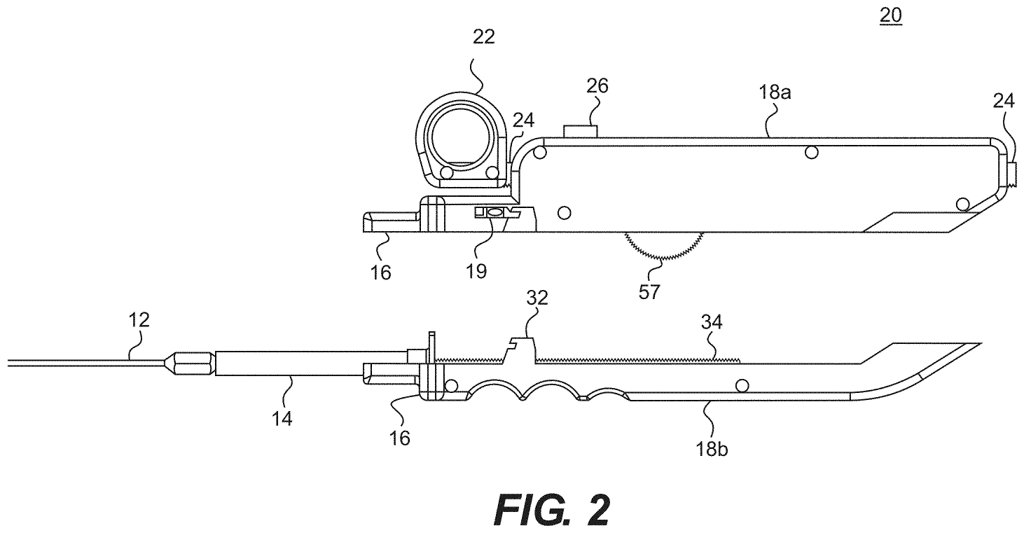

FIG. 2 is an illustration of a side view 20 of the exemplary injection control device of FIG. 1 with the upper body portion 18a and lower body portion 18b of the body 18 separated. Of note is the exposed latch engagement member 32 used for attachment to the latch 19 when the upper body portion 18a and lower body portion 18b are attached to each other. Also, FIG. 2 illustrates the lower portion of the exposed syringe rack gear 57 and the upper portion of the corresponding syringe rack 34. It should be appreciated that other forms of the latch engagement member 32 may be used than that shown in FIG. 2. That is, instead of latching with a slidable latch 19, a twisting or screwing, or otherwise engaging motion may be used with an appropriately designed latch engaging member 32, to achieve the desired securing operation, without departing from the spirit and scope of this disclosure. Therefore, other devices or mechanisms known in the art for securing the upper portion 18a and the lower portion 18b of the body 18 may be contemplated, according to design or efficiency preference.

Further, it should be appreciated that the exemplary embodiment shown in FIG. 2 may also be configured so that the body 18 is separated into a different configuration, such as to be arranged in “left” and/or “right”, or other arrangements, as opposed to “upper” and/or “lower”, etc. Therefore, it should be apparent that other shapes, whether paired or multiplied, or separation methodologies ranging from sliding, twisting, screwing, snapping, etc., for example, may be used to enable the user to access the interior of the exemplary injection control device. It should also be appreciated that in some embodiments, a gripping portion may be provided on the surface of the body 18 to enable a user a secure hold of the exemplary injection control device.

Additionally, while the exemplary injection control device is shown in FIG. 2 with a body 18 that may be separated, it is contemplated that a uni-body implementation may be used. That is, the body 18 may be formed as a single piece, not separable wherein the syringe 14 is “attached” to the body 18. Thus, a single body configuration may be made without departing from the spirit and scope of this subject matter.

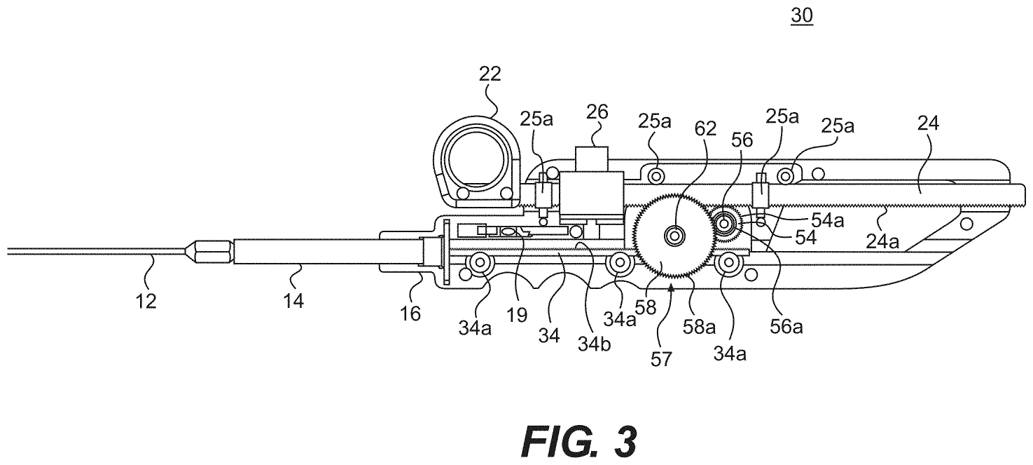

FIG. 3 is an illustration of an axial cut-away view 30 of the exemplary injection control device of FIG. 1. The cut-away view 30 reveals an exemplary gearing arrangement suitable for accomplishing at least one of the goals of the exemplary injection control device. For example, using the gearing arrangement shown in FIG. 3, it should be apparent to one of ordinary skill in the art that during the operation of the exemplary injection control device, as the ring 22 is fixed in place and the body of the injection control device is moved to the “right,” the syringe rack 34 will move to the “left”—acting as a plunger into the syringe 14 being held in the syringe supporting section 16. Therefore, any material in the syringe 14 will be expelled into the cannula 12. Based on appropriate gearing ratios of the exemplary gearing arrangement, a very precise and controlled injection of the filler material can be accomplished, with minimal technical expertise.

In an exemplary embodiment of the injection control device, the gearing arrangement of FIG. 3 is illustrated with the primary components of the positioning rack 24, engaging a positioning rack gear assembly 55. The positioning rack gear assembly 55 having an outer gear 54 and inner gear 56 and clutch (not seen) is coupled to a syringe rack gear 57 having an outer gear 58 and an inner gear 62 (not seen), which is engaged to the syringe rack 34. The positioning rack 24 is constrained and guided by positioning rack rollers/guides 25a, which are placed at strategic points along the travel area of the positioning rack 24, to guide and maintain smooth travel of the positioning rack 24 through the body 18. Similarly, syringe rack rollers/guides 34a are illustrated as guiding and/or constraining the syringe rack 34 within the body 18.

It should be appreciated that while FIG. 3 illustrates various rollers/guides 25a and 34a, disposed within and about the body 18, other forms or arrangements of rollers/guides that are known in the art or future-derived, may be used to achieve the desired effects, without departing from the spirit and scope of this disclosure. In fact, in some embodiments, the roller/guides 25a and 34a may be supplanted with full body guides along the body 18, such as a channel or sleeve. Since knowledge of such presently known rollers/guides and alternative arrangements are within the purview of one of ordinary skill in the art, they are not discussed herein.

In one mode of operation, the ring 22 is held stationary with respect to the subject's surface being “injected.” The body 18 of the injection control device is moved as the cannula 12 is withdrawn. In another mode of operation, it may be desirable to advance the entire injection control device as a unit as the cannula 12 is advanced into the tissue. Then the ring 22 is held stationary with respect to the tissue as the body 18 of the injection control device with the syringe 14 and cannula 12 is withdrawn expelling the injectate in the syringe 14. The ring 22 is then pushed back into the body 18 of the injection control device. The entire injection control device is then again advanced as a unit.

In another mode of operation, the reverse effect can be accomplished, wherein by advancing the cannula 12 into the tissue, material can be “sucked” into the injection control device. Therefore, as will be apparent from the description provided herein, multiple modes of operations may be contemplated, accordingly, the injection control device may also operate as a suction (extraction) control device.

In view of various movements of the body 18 with respect to the ring/positioning guide 22, the positioning rack's teeth 24a will engage with the teeth 54a of the outer gear 54 of the positioning rack gear assembly 55 and cause rotation. The positioning rack gear assembly 55 may be configured with teeth ratios to act as a reduction gear in order to translate the linear displacement of the positioning rack 24 to a reduced linear displacement of the syringe rack 34. As the teeth 56a of the inner gear 56 of the positioning rack gear assembly 55 engage with the teeth 58a of the outer gear 58 of the syringe rack gear 57, the teeth 62a (not shown) of the inner gear 62 (not shown) will engage the teeth 34b of the syringe rack 34, causing a linear displacement of the syringe rack 34.

It is should be apparent from the above description concerning the operation of the ICD that the ring 22, when held against a surface, etc., operates to “fix” the position of the end of the ICD and also, via its fixed connection to the positioning rack 24, forms a stationary reference point for the ICD's internal mechanics to react against. That is, the now “fixed” position of the positioning rack 24, being acted against by the ICD internal mechanics, facilities the conversion of the translation forces of the body 18 to motion of the syringe rack 34. It should also be apparent that since the syringe 14 is fixed to the body 18, as the body 18 is being translated the syringe's cannula 12 will also translate with the body 18. Consequently, as the cannula 12 is being translated in or out of the subject, the exemplary ICD injects or extracts in synchronicity with the cannula's 12 movement. Thus, injection or extraction occurs while the cannula 12 is moving.

Regarding terminology, since the ring 22 can extend to, or in some embodiments, beyond the tip of the cannula 12, it can function as a positionable member to assist in aligning the cannula 12 to the patient or subject. Also, since the positioning rack 24 is fixed to the ring 22, the combination of the ring 22 and the positioning rack 24 operates as a reference member for the internal transmission (e.g., gearing assembly, etc.) to react against as the body 18 is translated when the ring 22/positioning rack 24 is stationary or fixed. Accordingly, it is understood the term “positioning guide” as used herein does not describe a member that solely operates for positioning an injection device, but a member that is extendable to a fixed location (positionable) on the subject tissue, and being fixed provides a reference point or fixture for the body and associated transmission to react. Therefore, while the term “positioning guide” is used throughout this disclosure, it is expressly understood that it describes a positionable transmission reference member.

In an exemplary embodiment of the injection control device, a ratio of approximately 5.2093:1 was used to effect the desired movement of the positioning rack 24 with respect to the syringe rack 34. That is, for every 5.2093 inches the injection control device is displaced or “withdrawn” from the tissue with the ring 22 held in place, the syringe rack 34 advances approximately 1 inch. Given a commercially available 1 cc syringe, the exemplary injection control device will inject approximately 0.00436 cubic inches of filler material for every one inch the cannula 12 is withdrawn from the tissue.

The gearing ratio described above may be adjusted according to methods and systems known in the art of gearing. Therefore, the gearing ratio may be adjusted by simply replacing the appropriate gears and racks to achieve a desired injection rate. In such embodiments, a “dialing” in of a different gear ratio may be contemplated, according to gearing systems known in the art. Alternatively, to achieve a different or variable injection rate, varying syringes with different bore diameters may be used, to increase or decrease the rate of material injected. If the outside diameter of the syringe is held constant while the internal diameter is varied, this will allow the effective gear ratio or “injection rate” to be easily varied according to the application. This can prove to be a very economical way of “changing gears” without changing the actual gearing of the injection control device or switching to a similar injection control device with a different gear ratio.

As is made apparent from the above description, one mode operation of the exemplary injection control device may entail the user positioning the injection control device with the ring 22 (operating as a positioning guide) against the tissue or a pre-determined distance from the tissue. With the ring 22 (positioning guide) held in a stationary position, the body 18 of the injection control device can be advanced into the tissue and then withdrawn, with the ring 22 (positioning guide) held in place. Consequently, the advancing motion of the cannula 12 will create a tract in the tissue, while the withdrawing motion of the cannula 12 (the body 18 of the injection control device) will deposit the filler injectate in the void created in the tract as the cannula 12 is withdrawn.

In order for the ring 22 to be fixed at a desired position in proximity to the surface of the tissue, the ring 22 should be allowed to be manipulated in a “forward” or “skin”-side direction without causing the syringe rack 34 to move. This freedom is achieved by a clutching mechanism that is discussed in further detail below.

It should be appreciated that, in some embodiments, it may be desirable to have the ring 22 (positioning guide) flush to the surface of the tissue, thus providing the stable reference of the body surface for the user to exert a “push” against while he is “pulling” the injection control device. Of course, it should be apparent that depending on the preferences and skills of the user, the ring 22 may not placed against the tissue or surface but at a preferred distance. For example, a user may place his thumb into the ring 22 and use the span of his hand with his fingers or palm against the surface of the tissue, resulting in the ring 22 being positioned a pre-determined distance from the surface of the tissue. Thus, it should be apparent that variations of the placement of the ring 22 as well as its shape may be practiced without departing from the spirit and scope of this disclosure.

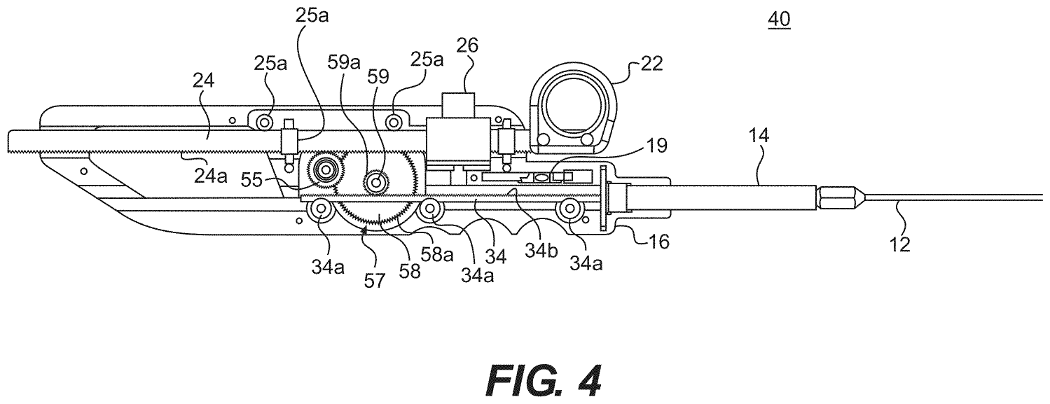

FIG. 4 is a close-up illustration 40 of the reversed side of the interior of the exemplary injection control device. FIG. 4 illustrates the teeth 59a of the syringe rack gear 57 engaging the teeth 34b of the syringe rack 34.

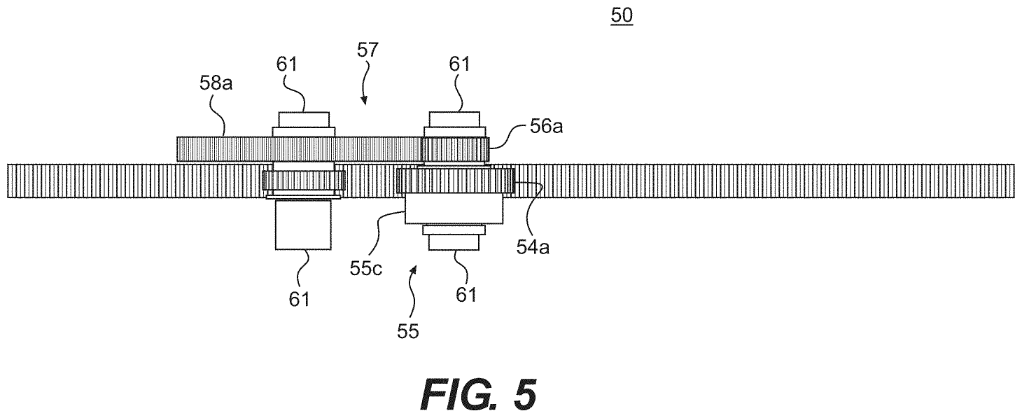

FIG. 5 is a bottom-side illustration 50 of the gear contacts of the exemplary injection control device with the syringe rack 34 removed from view. The positioning rack gear assembly 55 is shown with a clutch 55c which acts as an intermediary between the outer gear 54 and the inner gear 56 of the positioning rack gear assembly 55. The clutch 55c functions to provide a mechanism to enable “free” movement of the positioning rack 24 without causing the inner gear 56 of the positioning rack gear assembly 55 to move. Thus, the positioning rack gear may be moved in a preferred direction without causing the syringe rack gear 57 to turn. In principle, the clutch 55c allows advancement of the syringe plunger into the syringe cylinder but not its withdrawal. Therefore, the clutch 55c allows the exemplary injection control device to be advanced relative to the ring 22 without causing the plunger to move relative to the syringe cylinder.

As shown in FIG. 1, the brake 26 may be used to stop or engage the motion of the positioning rack 24. Therefore, by engaging the brake 26, the ring 22 may be secured while the cannula 12 is positioned in the tissue. It should be noted that the brake 26, in some embodiments may not be necessary, as operation of the injection control device can conceivably be executed without use of the brake 26.

In particular, the use of a clutch 55c or one-direction-engagement mechanism enables the user to adjust the position or extension of the positioning rack 24 from the body 18, with the ring 22 at a desired distance from the tissue, without causing the syringe rack 34 to move in a reverse orientation. The clutch 55c can be engaged in such a manner to cause the gear train to rotate and advance the syringe rack 34 (or plunger) into the syringe, as the body 18 of the injection control device is moved away from the ring 22. The clutch 55c allows the body 18 of the injection control device to move towards the ring 22 without the syringe rack 34 moving with respect to the syringe. Also, the clutch 55c can be configured to prevent the gear train from moving the syringe rack 34 with respect to the syringe as the body 18 is advanced with respect to the ring 22.

In some embodiments, the clutch 55c may be supplanted with an arrangement wherein the teeth 54a of the outer gear 54 are displaced from the teeth 24a of the positioning rack 24, by some switch or motion (not shown) that is coupled to the positioning rack gear assembly 55. Thus, by removing contact of the teeth 54a of the outer gear 54 from the teeth 24a of the positioning rack 24, the positioning rack 24 may be moved without causing the syringe rack 34 to move.

It should be appreciated that one of ordinary skill in the art of gearing may devise an alternative scheme for providing “free” movement of the positioning rack 24 in a preferred direction, or even in both directions. The above clutching mechanism 55c is provided as one simple scheme for achieving the desired results wherein more complicated or different schemes may be contemplated. Therefore, other schemes or systems for providing controlled motion or contactless motion may be used, whether using gears, clutches, slips, discs, springs, etc., without departing from the spirit and scope of this disclosure.

FIG. 5 also illustrates the use of gear axle caps 61 for the positioning rack gear assembly 55 and the syringe rack gear 57. It should be appreciated that in some embodiments, the gear axle caps 61 may not be necessary, as axle securing methods not consisting of caps 61 may be used, such as those that are common in the industry. Additionally, the illustrated spacing between the gears and rack(s) shown may be adjusted according to design preference.

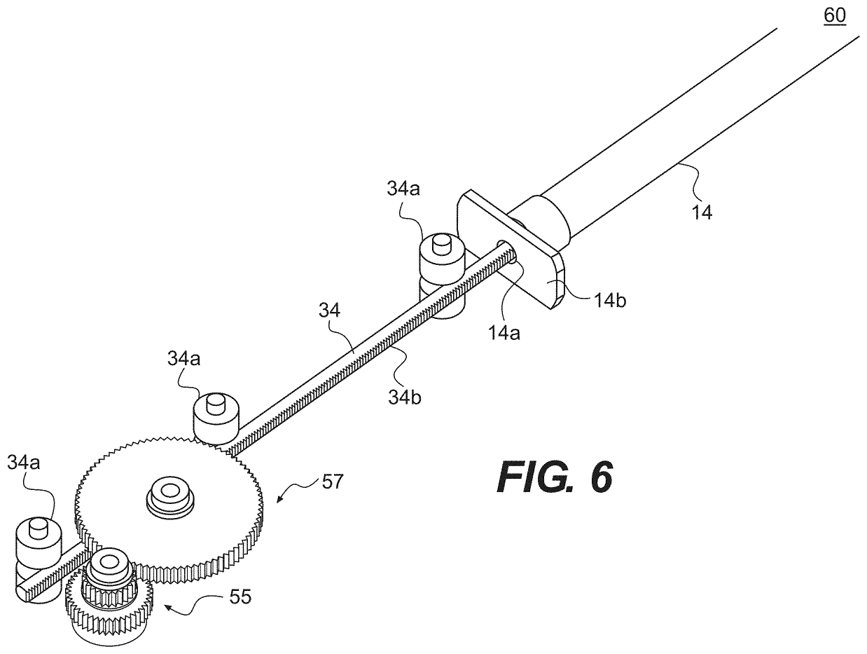

FIG. 6 is a perspective view illustration 60 of the syringe rack arrangement. Specifically, the syringe rack 34 is illustrated with a smooth ridge 34b that fits within a channel within the roller/guides 34a. By use of the smooth ridge 34b within the channel, lateral movement of the syringe rack 34 can be minimized. Of course, in some embodiments, the roller/guides 34a may be replaced with bearings, if desired. Or, the ridge 34b may be replaced with a channel “under” the syringe rack 34, wherein bearings or roller/guides may be disposed. In some embodiments, the syringe rack 34 may have a different shape, according to design preference. Therefore, round, square, rectangular or other shapes may be used. Also, a non-bearing configuration, using for example, the interior of the body 18 as a constraining and guiding entity may be used. Therefore, alternative arrangements for guiding the syringe rack 34 may be used without departing from the spirit and scope of this disclosure.

The syringe rack 34 is also shown in FIG. 6 as having its “front” plunger end inside an opening 14a of the syringe 14. In some embodiments the syringe rack 34 may be configured to drive another mechanism that acts as a plunger for the opening 14a of the syringe 14. Thus, some form of pivoting may be designed to cause the syringe rack 34 to move “outside” the opening 14a, while still achieved the desired effect of moving a plunger into or out of the syringe 14. In some embodiments, the syringe rack 34 may be an integral part of the syringe 14. That is, the syringe rack 34 may constitute the actual plunger mechanism in the syringe, or a controlling member. Thus, a syringe 14 may be configured with a syringe rack 34 pre-configured for use with the injection control device. Alternatively, the syringe rack 34 may be configured with a geometry that is suitable for use with disposable syringes. Therefore, the injection control device may use disposable syringes or may use syringes having a plunger with a syringe rack 34 attached. Moreover, the ICD itself may be designed to be disposable after a single use, or single procedure.

It should be noted that in FIG. 6, the anterior end of the syringe 14 is shown having flanges 14c. The typical syringe 14 is understood to have such flanges 14c, and therefore, the exemplary injection control device exploits the presence of the flanges 14c by accommodating them in bulged areas of the syringe supporting section 16. In some embodiments, the syringes 14 may not have such flanges 14c, therefore an appropriate securing mechanism may be devised, such as a clamp or well, for example, for securing the syringe 14 to the exemplary injection control device. In such embodiments, the flanges 14c may be of a reduced size and therefore, the upper body 18a and lower body 18b portions surrounding the flanges 14c may be altered in a manner suitable for achieving the desired effect, without departing from the spirit and scope of the disclosure

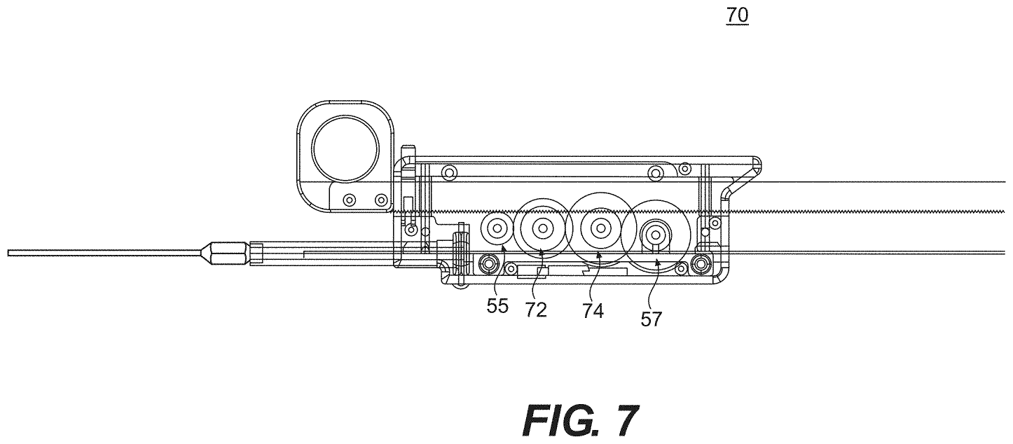

FIG. 7 is an illustration 70 of the outline of an exemplary injection control device with multiple gears. Specifically, the exemplary injection control device is illustrated with four gears, chaining action from the first positioning rack gear assembly 55 to a series of “reduction” gears 72 and 74, to the syringe rack gear 34. By use of multiple gears 72 and 74, varying amounts of ratios can be achieved. Of course, while FIG. 7 illustrates a total of four gears in the gear train, more or less gears may be used according to design preference.

By use of the exemplary injection control device several advantages can be obtained:
The injection of the injectate is substantially proportional to the length of the injection tract and uniform along the course of the injection tract;
An “automatic” controlled injection system can be used for injection;
A fixed amount of injectate can be injected per unit distance traveled by the tip of the cannula;
The injection ratio (amount of material injected over a given distance of cannula withdrawal) can be varied by simply using varying bore diameter syringes;
The use of syringes (disposable); and
The use of syringes incorporating a rack in the plunger.

It should be appreciated that based on an understanding of the exemplary injection control device disclosed herein, several modifications may be contemplated without departing from the spirit and scope of this disclosure. As some cannulas may be of different diameters and openings, a volume approach may be achieved by adjusting the gearing, for example.

As another modification, the clutch 55c may be configured to operate in a “reverse” manner than described. That is, rather than having the exemplary injection control device inject filler material, the exemplary injection control device may be configured to “suck” material. Thus, in some applications, harvesting material may be accomplished by altering the clutching or gearing of the exemplary injection control device.

Along the lines of the above modification, it is possible to design a gearing system that injects material as the cannula is advanced, rather than withdrawn. Additionally, the exemplary injection control device may be configured with opposing gear trains that would enable the injection of filler material as the cannula is advanced as well as when the cannula is withdrawn. Similarly, the exemplary injection control device may operate in a manner to enable the withdrawal or sucking of material as the cannula is advanced as well as when the cannula is withdrawn.

Several other variations of the exemplary injection control device described above are detailed below.

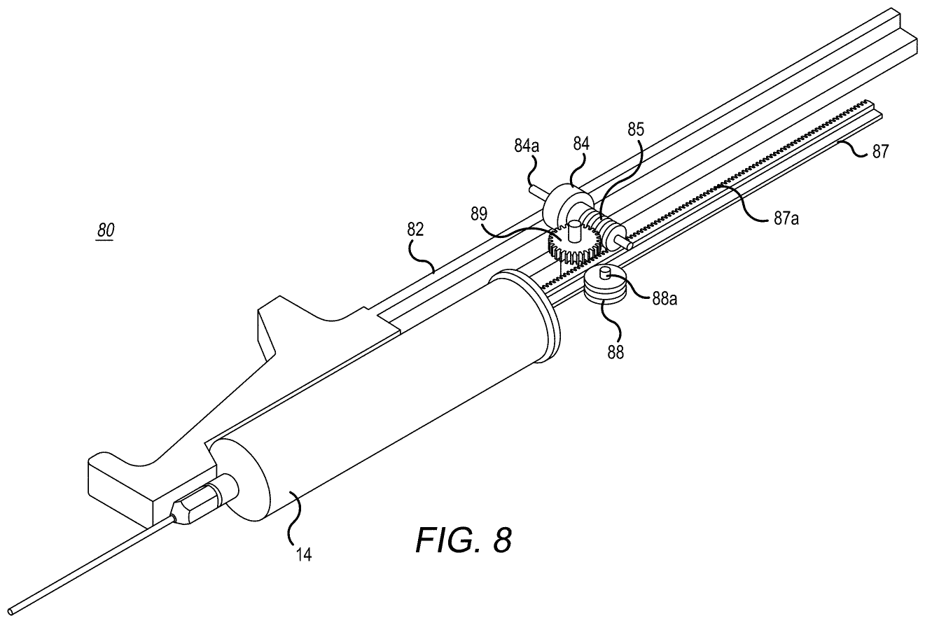

FIG. 8 is an illustration 80 of a perspective bodiless view of a “rackless” exemplary injection control device according to a second embodiment. The body is removed from view so the internal mechanisms can be seen, recognizing that syringe 14 is fixed to the removed body. The general principles of operation are similar to the previous embodiment, but with a rackless positioning guide 82. In this FIG. the syringe-side of rackless positioning guide 82 is also shown in close proximity to the body of syringe 14, shielding one side of the syringe 14. The rackless positioning guide 82 is achieved by use of a spooling mechanism 84 coupled to rackless positioning guide 82 with concentric worm gear 85 that engages main gear 89. Spooling mechanism 84 contains a clutch or locking/unlocking mechanism (not shown) controlling worm gear's 85 ability to rotate with spooling mechanism 84. Roller bearings used to support the positioning guide in the previous embodiment can be replaced by sleeve bearings (not shown) in the removed body. Axle 84a of spooling mechanism 84, axle 89a of main gear 89, axle 88a of bearing 88, and syringe 14 are secured to removed body, so that these elements travel with the body as the body is translated.

Thus, with the clutch is engaged, worm gear 85 rotates with spooling mechanism 84 as the removed body is translated with respect to rackless positioning guide 82. Spooling mechanism 84 will unwind, turning worm gear 85 which turns main gear 89, which engages teeth 87a of plunger rack 87 to drive or retract the stopper (not shown) in the syringe 14. Plunger rack 87 may be supported by a single bearing 88. When the clutch is not engaged, spooling mechanism 84 may rotate without causing rotation of worm gear 85. It is noted it is possible that the resistance of the stopper in syringe 14 will operate to obviate the need for a second clutch to prevent movement of the plunger rack 87 during preliminary setup of the ICD.

Spooling mechanism 84 may be a drum with a coil or a constant force spring, for example. Coil portion (end of) the constant force spring can be attached to the forward or aft section of rackless positioning guide 82, depending on the mode of operation. The constant force spring provides tension on the coil to allow it to wind properly. Further, if enough tension is provided, the winding force may be sufficient to assist in driving (or withdrawing—depending on mode of operation) the plunger rack 87 back to its starting position. For injection/extraction materials that are particularly viscous or thick, the implementation of an assistive device (such as the constant force spring) can be beneficial. It is envisioned, in some embodiments the positioning guide 82 may start in the extended position and via insertion of the cannula 12 into the tissue, the insertion force operates to push the positioning guide 82 into the retracted position and loads the constant force spring, which in turn provides the motive force to drive the gear train, as the positioning guide 82 is allowed to push the body of the ICD away from the tissue being injected. Accordingly, a constant force spring or spring motor could be loaded by a winding mechanism to store energy that could be used to assist in the injection.

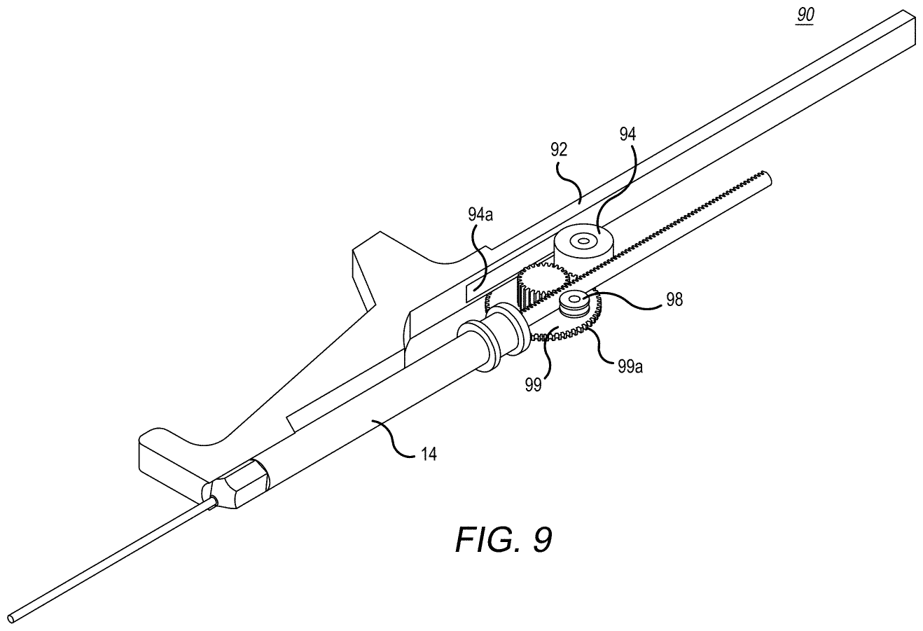

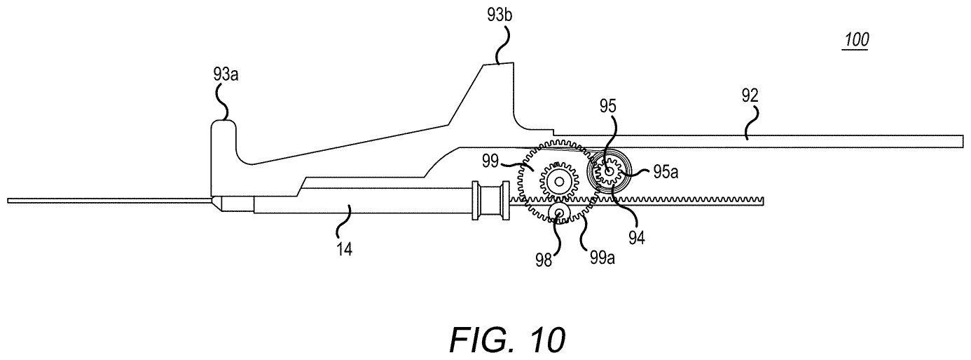

FIGS. 9-10 are illustrations of another bodiless “rackless” exemplary injection control device according to a third embodiment. FIG. 9 is an illustration 90 of a perspective view and FIG. 10 is a side cut-way view 100. These FIGS. show a variation of the embodiment shown in FIG. 8. As seen in FIG. 9-10, instead of using a worm gear, spooling mechanism 94 (or constant force spring, for example) is joined with a coaxial gear 95 having teeth 95a that directly contact main gear 99 via main gear's outer teeth 99a, to drive main gear 99. In this embodiment, constant force spring is used as the spooling mechanism 94 and is aligned in the same orientation as the main gear 99 to obviate the need for a worm gear as well as reduce the overall “thickness” of the gearing assembly. Coil of the constant force spring is attached 94a to a side of rackless positioning guide 92. The subsequent mechanics of motion for operation of the ICD are similar to those described in FIG. 8.

It is worthy to note in passing that similar to FIG. 8, the forward portion of the positioning guide 92 is configured without a “thumb” or “finger” hole, but is configured with two open extensions 93a and 93b. The openness of these extensions allows them to be pressed against using a palm or fingers. For example, extension 93a can be “pushed” forward using a palm resting against the extension, while extension 93b can also be “pushed” forward via a palm or fingers. Conversely, extension 93b can be large enough to be gripped with a hand to be “pulled” back (i.e., retracted), if the mode of operation requires such a motion. A certain increased ease of handling is obtained by having open extensions versus a closed extension (thumb or finger hold such as seen in the first embodiment). While palm, fingers, hands are described as pulling or gripping to cause the desired motion, it is understood that the exemplary injection control device may be actuated using other means and ergonomics including but not limited to a trigger squeeze mechanism or a hand squeeze mechanism.

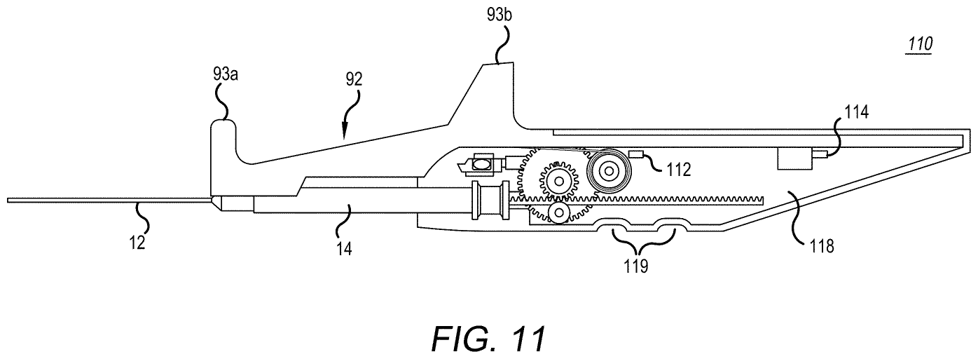

FIG. 11 is an illustration 110 of an internal side cut-away view of the exemplary injection control device of FIGS. 9-10 but with body 118 and one or more adjustable stops 112, 114. The adjustable stops 112,114 operate to limit the range of motion for the body 118 and/or the positioning guide 92 as it slides through body 118. This “control” effectively limits the distance the cannula 12 will travel with respect to the positioning guide 92. Use of the adjustable stops 112, 114 will precisely control the distance over which the deposition/extraction of material occurs.

The adjustable stops 112, 114 can be a pin that is inserted in multiple accommodating “holes” (not shown) along the body/positioning guide or a sliding lock as seen in disposable box cutters. Of course, other forms or mechanisms for locking or restricting the range of motion may be used and are understood to be within the purview of one of ordinary skill.

Also evident in this FIG. is that, for this example, the front 93a of positioning guide 92 has been designed with a substantially flat surface, thus conceivably acting as a “depth” gauge—preventing insertion of the cannula 12 past a certain point on the cannula 12. In regard to gauges, this or other exemplary ICDs may have a gauge (not shown) external or visible on the body 118, to allow the user to view the amount of material in the syringe 14. In some embodiments, the body 118 may have an opening (not shown) that allows viewing of the syringe 14. To this end, body 118 does not completely encase syringe 14, as in the first embodiment. Rather the bulk of the syringe 14 is exposed, which allows for the user the ability to visually inspect the syringe's contents, before and after administration. As in previous embodiments body 118 is configured with optional finger rests 119.

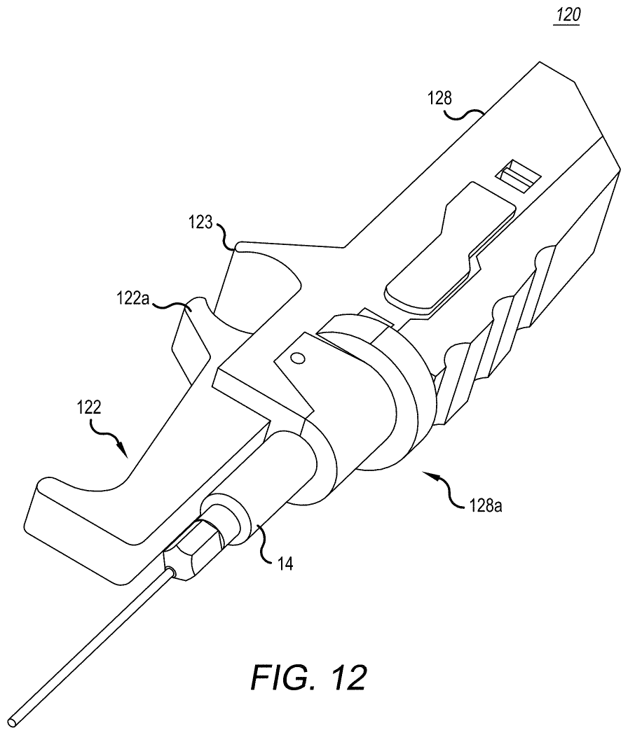

FIG. 12 is an illustration 120 of a perspective view of an exemplary injection control device according to a fourth embodiment. The body 128 is shown with a thumb rest 123 to assist in gripping the ICD as the cannula 12 is advanced into the tissue. Thumb rest 123 can also operate as safety ridge to reduce accidental pressure on the exposed rear portion 122a of positioning guide 122. Thumb rest/safety ridge 123, depending on design preference, can also operate as a fixed stop for positioning guide 122, restricting the amount of retraction available to positioning guide 122. Body 128 can also be accommodated with a separable lower portion 128a that allows for access to the interior of body 128—so as to insert the syringe 14 into the ICD. In some embodiments, the body 128 can be configured into a clamshell design where the bottom half rotates on an axis located in the end of the device away from the cannula 12.

FIG. 13 is an illustration 130 of another exemplary injection control device according to a fifth embodiment, designed for automatic or near-automatic refilling. This device has cannula-side end 134 with a large pistoned chamber 139 connected to an outside feed line (not shown) via intake valve 135, allowing for refilling of the syringe without having to remove the syringe from the ICD. Intake valve 135 is configured to only allow flow into the pistoned chamber 139 from an outside injectate container. This enables rapid and repeated use of the ICD. For example, as a meat injector for various roasted or cooked meats. Hence, the injectate (not shown) would be a marinade or flavoring, etc. FIG. 13 also shows ICD body 138 and position guide 132 (in a closed position). It should be noted that in this illustration 130, the syringe is not fitted with a cannula and the pistoned chamber 139 is shown with no injectate inside. In a ready-for-use scenario, a cannula would be attached and cannula-side end 134 and the pistoned chamber 139 would contain the injectate or marinade.

It should be noted, that as a design choice valve 135 is considered a one-way valve or unidirectional valve, for ease of operation. However, it may be possible to have “switchable” bi-directional valve. That is, in a less elegant design, the “injection” operation of the ICD would have a switchable bi-directional valves in an injection-mode configuration (e.g., valve 135 closed to prevent injectate from flowing out of valve 135), via a switch or lever or button on the valve. And when in refill-mode operation, valve 135 would be flipped over to the opposite flow (e.g., valve 135 opened to allow injectate flow into the pistoned chamber 139).

In another design choice, cannula-side end 134 may be replaced or added to with an output valve that provides one-way or unidirectional flow of the injectate into the subject tissue (i.e., meat) whilst preventing “return” flow of air via the cannula (not shown) when the pistoned chamber 139 is being refilled via intake valve 125. This option is for situations where the cannula is of a large diameter and air would easily be sucked into the pistoned chamber 139, negating the drawing of the refill injectate. As in the previous discussion, the output valve may also be a switchable bi-directional valve.

The ICD transmission movement mechanics of this design are similar to one or more of the previous designs, however, the “barrel” of the syringe contains the large pistoned chamber 139 and principal flow and refilling is controlled intake valve 135. The pistoned chamber 139 can be considered a “reservoir” for the injectate that is supplied from an external larger source.

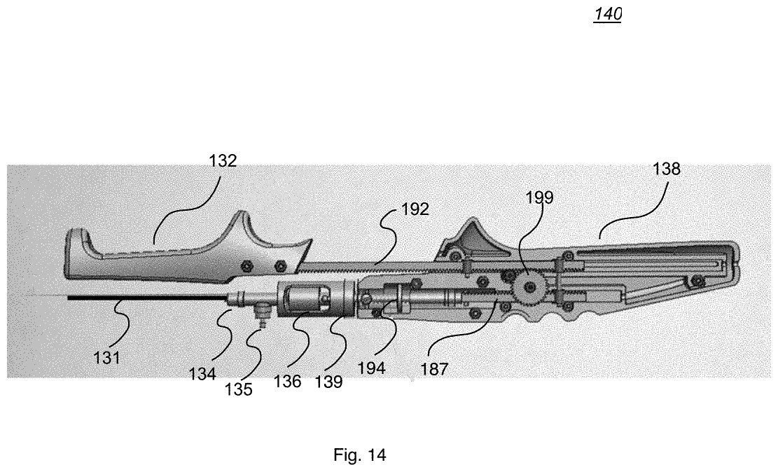

FIG. 14 is a side cut-away view of the embodiment of FIG. 13, shown in an extended state (post-use) with a cannula 131 attached directly or indirectly to the cannula-side end 134. In the post-use state, positioning member 132 is shown almost fully extended from the body 138 with a racked positioning guide 192, engaging main gear 199. In this example, piston 136 within pistoned chamber 139 is shown also shown as almost fully extended into the pistoned chamber 139. In the extended (post-use) state, the positioning member 132 is tensioned with a spring 194 or equivalent retraction providing mechanism that automatically causes the positioning member 132 to retract back, upon release of the positioning member 132. When retracting back, the main gear 199 (via one or more secondary gears) causes the plunger rack 189 to retract also, thereby causing the piston 136 to retract within pistoned chamber 139. The retracting piston 136 produces a vacuum within the pistoned chamber 139, which in turn causes the pistoned chamber 139 to automatically refill with the injectate via intake valve 135 (which is presumed to be connected to a refill canister or refill tube), thus preparing the ICD for the next use.

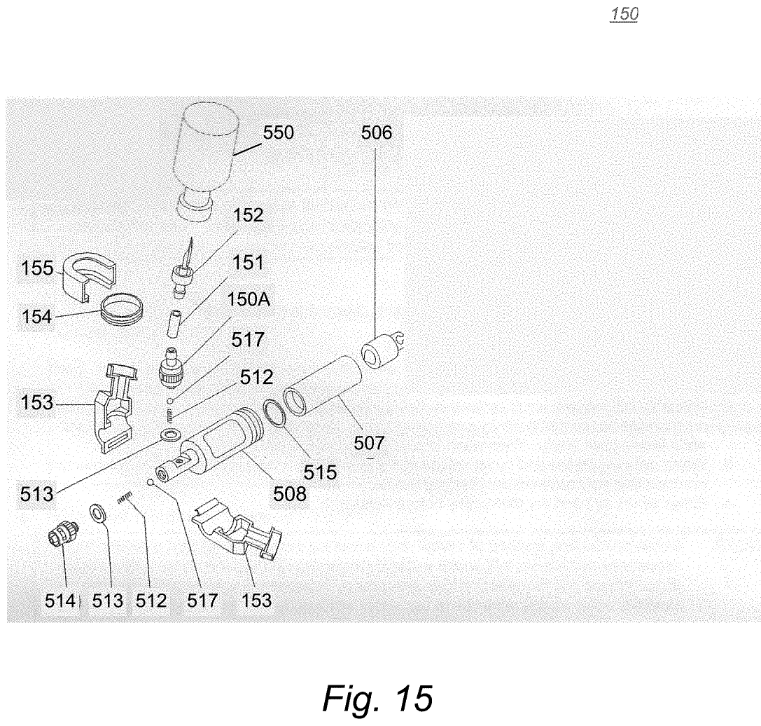

FIG. 15 is a blow up illustration 150 of the mechanics of a commercially available pistoned chamber and refill canister system suitable for the exemplary ICD of FIGS. 13-14. The system when configured with an ICD operates to provide rapid refilling. The example shown is from a manufacturer called Socorex, but other manufacturers may have similarly functioning systems that can be used with the exemplary ICD. For completeness, a description of the various elements and parts of the Socorex system, is given, noting that in other designs, more or less, as well as different, components may be used. Therefore, the example given for this system by Socorex is understood to not be limiting, as other similarly functioning systems can be used with the exemplary ICD.
508—Casing
515—Barrel casing washer
507—Glass barrel
506—Piston
153—Vial holder body
517—Valve ball
512—Valve spring
513—Valve washer
514—Nozzle
150A—Intake valve
151—Connection tube
152—Intake needle (vial)
154—Vial holder ring
155—Vial holder lock
550—Refill canister

It should be appreciated that some of the elements described in FIG. 15 may not be necessary for use or may be slightly varied to accommodate the ICD. For example, nozzle 514 will typically be replaced with a cannula holder to allow for attachment of the cannula. Additionally, vial holder lock 155 or vial holder ring 154 may be optional, etc.

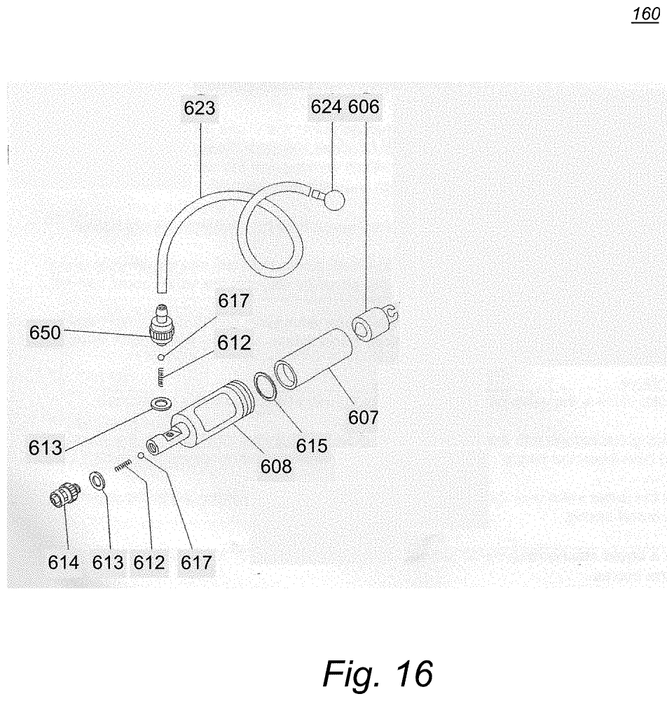

FIG. 16 is a blow up illustration 160 of another commercially available pistoned chamber with a refill tube system suitable for the exemplary ICD of FIGS. 13-14. The example shown is also from Socorex. For completeness, a description of the various elements and parts of the Socorex system is given, noting that in other designs (from other manufacturers), more or less, as well as different, components may be used. Therefore, the example given for this system by Socorex is understood to not be limiting.
608—Casing
615—Barrel casing washer
607—Glass barrel
606—Piston
617—Valve ball
612—Valve spring
613—Valve washer
614—Nozzle
650—Intake valve
623—Refill tube
624—Tube connector

As stated above, some of the elements described in FIG. 16 may not be necessary for use or may be slightly varied to accommodate the ICD. For example, nozzle 614 will typically be replaced with a cannula holder to allow for attachment of the cannula.

It should be appreciated that based on an understanding of various embodiments of the injection control device disclosed herein, several modifications may be contemplated without departing from the spirit and scope of this disclosure. As some cannulas may be of different diameters and openings, a volume approach may be achieved by adjusting the gearing, for example.

As one possible example, the valves described above may be of a disposable, limited use form, being able to be switched out without much effort, if so needed. Also, the intake valve may be positioned away from the body of the syringe such that there is feed line between the valve and the intake to the syringe. The valve would have an intake feed line from the reservoir of the injectate. So, the intake feed line may be connected to an intake that does not have an incorporated unidirectional valve. The valve may be positioned more “upstream” in a break in the feed line. “Upstream” can mean closer to the reservoir containing the injectate. The purpose of this modification would be to accommodate a larger unidirectional valve that is less prone to clogging when injecting fluids that are more viscous or contain significant amounts of particulate matter. As a larger unidirectional valve positioned close to the syringe intake may make the device awkward to maneuver close to the meat. Also, it is conceivable of using a pressurized reservoir to assist pushing the injectate through the tubing to the syringe. This approach also suggests a separate “pumping” system may be added to provide the pressure, as needed. In other possible embodiments, the pistoned chamber may not utilize a piston, so to speak, but another form of liquid-providing pressure. Non-limiting examples may be a rotating screw in the chamber pushing the injectate forward, a moving ball, etc. and etc. Thus, other forms of moving the injectate can be utilized, according to design preference.

In various applications, it is envisioned that using a cylindrical cannula will result in cylindrical tracks of material left in the channel created by the cannula's intrusion. Using computer/automated devices, an increased degree of control can be obtained in the amount and “shape” of the deposited material or extracted material as well as variation of the injection/extraction profile. For example, conical, elongated spheres, or series of spheres could be produced. A similar result can also be obtained by using a camming system in the transmission system to periodically delay/increase the rate of injection/extraction. Following this, a robotic system which precisely controls the position and rate of motion of the cannula and/or rate of injection/extraction could be implemented in the ICD. Moreover, while the “applications” are in the context of a cannula “inside” a subject, the ICD can be easily adapted to regulate the rate of extrusion of a fluid for a topical application.

It will be understood that many additional changes in the details, materials, steps and arrangement of parts, which have been herein described and illustrated to explain the nature of the disclosure, may be made by those skilled in the art within the principle and scope of the disclosure as expressed in the appended claims.

What is claimed is:

1. An injection control device, comprising:
a body having a syringe end and a gripping end;
a syringe barrel coupled to the body;
a refillable reservoir and at least one directional valve, disposed at the syringe end of the body, one of the at least one directional valves permitting refilling of the refillable reservoir without removing the syringe barrel; and
a transmission system coupled to the body, comprising:
a positioning member coupled to the transmission system and extendable from the syringe end of the body;
a syringe plunging member coupled to the transmission system; and
a gearing mechanism linking the positioning member to the syringe plunging member,
wherein when the positioning member is held in a fixed position and the body is pulled by the gripping end, the transmission system provides a one-way motion of the syringe plunging member to inject fluid from the device, a rate of the injected fluid being directly related to a rate of displacement of the body from the held positioning member.

2. The injection control device of claim 1, wherein the refillable reservoir is automatically refilled with injectate if the one of the at least one directional valves is connected to an injectate supply as the positioning member is returned to a resting, non-extended state.

3. The injection control device of claim 1, further comprising a cannula coupled to the syringe end.

4. The injection control device of claim 1, wherein the gearing mechanism comprises a plurality of gears, at least one gear contacting the positioning member and at least one other gear contacting the syringe plunging member.

5. The injection control device of claim 1, further comprising, a retraction mechanism coupled to the syringe plunging member or to a piston within the refillable reservoir, configured to provide a returning action to the positioning member upon its release from being held, wherein the returning action engages the transmission system to withdraw the syringe plunging member and automatically refill the refillable reservoir from injectate entering the one of the at least one directional valves.

6. The injection control device of claim 5, wherein the retraction mechanism is a spring.

7. The injection control device of claim 3, where another of the at least one directional valves is configured to prevent injectate from being drawn back into the refillable reservoir from the cannula.

8. The injection control device of claim 7, wherein the another of the at least one directional valves only provides flow from the refillable reservoir to the cannula.

9. The injection control device of claim 1, wherein the one of the at least one directional valves is displaced from the refillable reservoir and connected to a refill tube.

10. The injection control device of claim 1, further comprising a container of injectate connected to a refill tube connected to the at least one directional valve, wherein the container is either pressurized or non-pressurized.

11. The injection control device of claim 1, further comprising a refill vial, coupled directly or indirectly to the one of the at least one directional valves.

12. The injection control device of claim 1, wherein the at least one directional valves and switchable from bi-directional to unidirectional.

13. The injection control device of claim 1, further comprising a movable piston within the refillable reservoir and coupled to the syringe plunging member.

14. An injection control device, comprising:
a body having a syringe end and a gripping end;
a syringe barrel coupled to the body;
one or more injectate-refillable means disposed proximal to the syringe end of the body;
a flow control means coupled to the injectate-refillable means;
a transmission means coupled to the body, comprising:
a positioning means coupled to the transmission means and extendable from the syringe end of the body;
a syringe plunging means coupled to the transmission means; and
a linking means linking the positioning means to the syringe plunging means,
wherein when the positioning means is held in a fixed position and the body is pulled by the gripping end, the transmission means provides a one-way motion of the syringe plunging means to inject fluid from the device, a rate of the injected fluid being directly related to a rate of displacement of the body from the held positioning means.

15. The injection control device of claim 14, wherein the injectate-refillable is automatically refilled with injectate if the one of the at least flow control means is connected to an injectate supply as the positioning means is returned to a resting, non-extended state.

16. The injection control device of claim 14 wherein the linking means contacts the positioning means and the syringe plunging means.

17. The injection control device of claim 14, further comprising, a retraction means coupled to the syringe plunging means or to the injectate-refillable means, configured to provide a returning action to the positioning means upon its release from being held, wherein the returning action engages the transmission means to withdraw the syringe plunging means and automatically refill the injectate-refillable means from injectate entering the flow control means.

18. The injection control device of claim 14, another flow control means disposed at the syringe end of the body to prevent injectate from being drawn back into the injectate refillable means from a cannula.

19. A method for refilling an injection control device without replacing a syringe barrel, comprising:
coupling a refillable reservoir and at least one directional valve, to a syringe of an injection control device body having a syringe end and a gripping end;
coupling a transmission system to the body;
coupling a positioning member to the transmission system, the positioning member being extendable from the syringe end of the body;
coupling a syringe plunging member to the transmission system, the syringe plunging member be movable by the transmission system;
linking a gearing mechanism to the positioning member and to the syringe plunging member; and
refilling the refillable reservoir by retracting the positioning member, a motion of the positioning member operating on the transmission system to cause the syringe plunging member to retract from the refillable reservoir and create a suction to cause injectate to flow into the refillable reservoir from an external source coupled to the at least one directional valve.

20. The method of 19, further comprising coupling a retraction mechanism to at least one of the syringe plunging member and refillable reservoir, to automatically cause the positioning member to retract back to a non-extended state, wherein the refillable reservoir is filled from an external pressurized or non-pressurized injectate container.
